(19) United States
(12) Patent Application Publication
(10) Pub. No.: US 2011/0160741 A1
(43) Pub. Date: Jun. 30, 2011

(54) MEDICAL TREATMENT TOOL FOR TUBULAR ORGAN

(76) Inventors: Hiroyuki Asano, Kanagawa (JP); Gordon Hocking, Tokyo (JP)

(21) Appl. No.: 12/737,098

(22) PCT Filed: May 18, 2009
(86) PCT No.: PCT/JP2009/059115

§ 371 Date: Mar. 11, 2011

(30) Foreign Application Priority Data
Jun. 9, 2008 (JP) 2008-150952

(51) Int. Cl. A61B 17/22

(52) U.S. Cl. 606/127

(57) ABSTRACT

The invention is to provide a medical treatment tool for tubular organ which can surely capture or pick foreign substances such as calculi, other living tissues, etc. generated in a tubular organ or a body cavity and which can discharge or collect those foreign substances, other living tissues, etc. smoothly.

The medical treatment tool 10b for tubular organ includes: a tube 15; a wire 25 made of a shape memory alloy and inserted in the tube 15; a basket 30b formed into a cylindrical shape by knitting and/or braiding a plurality of metal wire rods 31, a base end portion thereof being bundled and connected to a tip end portion of the tube 15, a tip end portion thereof being bundled and connected to a tip end portion of the wire 25; and a handle portion 40 which holds a base end portion of the tube 15 and which holds a base end portion of the wire 25 to allow a relative movement to the tube 15.

### TECHNICAL FIELD

The present invention relates to a medical treatment tool for tubular organ, used for collecting or discharging foreign substances such as calculi or living tissues generated in a tubular organ or a body cavity.

### BACKGROUND ART

Calculi such as gallstones, pancreatic stones, etc. may be generated in tubular organs such as a bile duct, a pancreatic duct, etc. Various methods have been tried to remove these calculi from the tubular organs.

As one of such methods therefor, an endoscope is used. A crushing tube is inserted in a bile duct via an endoscope so that the crushing tube collides with the gallstone to thereby crush a massive gallstone generated in the bile duct into small fragments, for example. Then, a balloon catheter in a reduced diameter state is inserted into the bile duct via the endoscope and moved to a position beyond the region where the gallstone has been crushed, and water or air is injected into the catheter at that position to thereby inflate a balloon. Then, the catheter is pulled back to a proximal side to thereby scrape the crushed and scattered gallstone fragments so that the gallstone fragments are moved to the duodenum having a large inner diameter while discharged from the bile duct.

Various tools have been proposed as medical tools such as the aforementioned balloon catheter the diameter of which is widened and reduced in accordance with a treatment-target site. For example, Patent Document 1 discloses a basket type gripping forceps. In the basket type gripping forceps, an expandable/contractible basket portion is provided at a tip end of an operation wire. The operation wire is inserted in a sheathe so as to be movable forward/backward so that the basket portion is taken in and out from a tip end of the sheathe. In the basket type gripping forceps, the basket portion has groups of arranged elastic wires, and the elastic wires in each group have bending points in the same position at an intermediate portion from the tip end to the base end. By pulling the operation wire, the basket portion is retracted into the sheathe. By pushing the operation wire, the basket portion is extruded from the tip end of the sheathe.

Patent Document 2 discloses an emboli capture guide wire system including a core wire, a foldable emboli filter disposed on an outer circumference of the core wire, and a transmission sheathe provided for transmitting the emboli filter to an affected region while housing the emboli filter in the folded state at a distal end thereof. The emboli filter is made of Nitinol (Ni—Ti-based shape memory alloy) and a high-molecular membrane having pores is provided at the distal end.

In use, the emboli filter is folded and housed in the distal end of the transmission sheathe in advance, and the distal end of the transmission sheathe in this state is moved to a position beyond a treatment-target site such as an occluded portion. Then, by pulling the transmission sheathe back to the proximal side, the emboli filter is released from the distal end and expanded to its original size by the elasticity, shape memory effect, etc. of Nitinol, so that the emboli filter is lodged on the downstream side of the treatment-target site.

Patent Document 1: JP-H11-285500-A

Patent Document 2: JP-2007-216013-A

### DISCLOSURE OF THE INVENTION

### Problems that the Invention is to Solve

As described above, when fragment-like gallstones are discharged so as to be scraped by an inflated balloon catheter, resistance for passing the balloon catheter through a narrowed portion (e.g. a root portion etc. connected to the duodenum) of a bile duct may be so large that the gallstones can be hardly discharged.

In the basket type gripping forceps disclosed in Patent Document 1, the sheathe is essential to the operation of reducing and widening the diameter of the basket portion because the basket portion has a predetermined shape. As a result, it is necessary to insert the basket portion up to a use position in a state where the basket portion is housed in the sheathe. If the lumen of the tubular organ is occluded or clogged with thrombi, calculi, etc., there is a possibility that the calculi, etc. cannot be captured by the basket portion because it is impossible to pass the sheathe.

In the emboli filter disclosed in Patent Document 2, the diameter of the emboli filter is reduced when the emboli filter is housed in the distal end of the transmission sheathe, and the diameter of the emboli filter is widened when the emboli filter is released from the distal end of the transmission sheathe. The transmission sheathe is required for the operation of widening and reducing the diameter of the emboli filter. For this reason, when, for example, the lumen of the tubular organ is occluded or clogged with calculi, etc., there is a possibility that thrombi, etc. cannot be captured because it is impossible to pass the transmission sheathe through the lumen so that the diameter of the emboli filter cannot be widened in a predetermined position in the same manner as in Patent Document 1.

Therefore, an object of the invention is to provide a medical treatment tool for tubular organ, which can surely capture or pick foreign substances such as calculi, other living tissues, etc. generated in a tubular organ or a body cavity and which can discharge or collect those foreign substances, other living tissues, etc. smoothly.

### Means for Solving the Problems

To achieve the foregoing object, according to a first aspect of the invention, there is provided a medical treatment tool for tubular organ, including: a tube; a wire made of a shape memory alloy and inserted in the tube; a basket formed into a cylindrical shape by knitting and/or braiding a plurality of metal wire rods, a base end portion thereof being bundled and connected to a tip end portion of the tube, a tip end portion thereof being bundled and connected to a tip end portion of the wire; and a handle portion which holds a base end portion of the tube and which holds a base end portion of the wire to allow a relative movement to the tube.

According to the invention, when the handle portion is operated to push the wire to the axial-direction tip end side relative to the tube, the wire is extruded from the tip end of the tube. Accordingly, the basket having the base end portion connected to the tip end portion of the tube and the tip end portion connected to the tip end portion of the wire is expanded in the axial direction and clustered together as a whole so that the diameter of the basket is reduced. On the other hand, when the handle portion is operated to pull the wire to the axial-direction base end side relative to the tube, the portion of the wire protruding from the tip end of the tube is pulled into the tube. Accordingly, the basket is pressed and contracted in the axial direction so that the diameter of the axial-direction intermediate portion of the cylindrical basket is widened in the circumferential direction.

Usage of the medical treatment tool will be described. The wire is pushed to the axial-direction tip end side relative to the tube to reduce the diameter of the basket. In this state, the medical treatment tool is housed in a medical tube such as a catheter. For example, the catheter is inserted in a tubular organ such as a bile duct or a pancreatic duct or in a body cavity so that the catheter reaches a position just before the region where foreign substances such as gallstones or pancreatic stones are present. Then, the medical treatment tool is pushed to the axial-direction tip end side relative to the catheter, so that the diameter-reduced basket is protruded from the tip end of the catheter and moved to a position beyond the foreign substances. In this state, the wire is pulled out to the axial-direction base end side relative to the tube, so that the portion of the wire protruding from the tip end of the tube is pulled into the tube to thereby widen the diameter of the basket. When the medical treatment tool is moved to the proximal side while the diameter of the basket is widened in this manner, the foreign substances can be moved to a relatively large lumen portion such as the duodenum and discharged. When the foreign substances are captured by the basket and the medical treatment tool is pulled out together with the catheter from the tubular organ or the like, the foreign substances can be also collected.

Even when the lumen of a tubular organ or the like is filled with foreign substances so that a gap is small, the diameter of the basket can be widened and reduced desirably by the sliding operation of the wire. Accordingly, even when the gap of the lumen of the tubular organ or the like is small as described above, the diameter of the basket can be reduced suitably so that the basket can be passed through the lumen easily and then the operation of discharging the foreign substances can be performed surely.

Moreover, because the basket is formed into a cylindrical shape by knitting and/or braiding plural metal wire rods, the basket has flexibility compared with a balloon inflated with water, air, etc. As a result, even when the basket is fitted to the inner circumferential shape of the tubular organ so that a gap is hardly generated and the basket passes through a narrow portion when the medical treatment tool is pulled back after the foreign substances are captured by the basket, diameter-widening force of the basket can be kept while the diameter of the basket is reduced suitably in accordance with the inner diameter of the narrow portion. Accordingly, resistance for pulling out the basket can be reduced, and the foreign substances can be discharged surely and smoothly.

According to a second aspect of the invention, there is provided the medical treatment tool of the first aspect of the invention, wherein metal wire rods of the basket at a side of the base end portion are formed into plural bundle portions each formed by bundling plural metal wire rods, and wherein the bundle portions are jointed to the tip end portion of the tube so that opening portions are formed between the bundle portions.

According to the invention, when the wire is pulled out to the axial-direction base end side relative to the tube so that the portion of the wire protruding from the tip end of the tube is pulled into the tube to thereby widen the diameter of the basket, the bundle portions are separated from each other widely so that relatively large opening portions are formed on the base end side of the basket. Accordingly, when the diameter-widened basket is moved to the proximal side after the basket passes through foreign substances, the foreign substances can be captured into the basket through the opening portions. Accordingly, the foreign substances can be captured surely.

Accordingly to a third aspect of the invention, there is provided the medical treatment tool of in the first aspect of the invention, wherein the metal wire rods of the basket at a side of the tip end portion are formed into plural bundle portions each formed by plural metal wire rods, and wherein the bundle portions are jointed to the tip end portion of the wire so that opening portions are formed between the bundle portions.

According to the invention, when the wire is pulled out to the axial-direction base end side relative to the tube so that the portion of the wire protruding from the tip end of the tube is pulled into the tube to thereby widen the diameter of the basket, the bundle portions are separated from each other widely so that opening portions are formed on the tip end side of the basket.

For example, because foreign substances are excessively captured into the basket so that the basket can hardly pass through the narrowed region of the tubular organ or the basket can be hardly opened and closed, the foreign substances excessively captured into the basket may be hardly discharged to a tubular organ large in lumen. Even in this case, in accordance with the invention, the foreign substances excessively captured into the basket can be discharged from the opening portions because the opening portions are provided on the tip end portion side of the basket. As a result, the basket can be passed through even the narrowed region of the tubular organ smoothly, and the operation of discharging the foreign substances from the basket can be performed easily in the tubular organ large in lumen.

According to a fourth aspect of the invention, there is provided the medical treatment tool of any one of the first to third aspects of the invention, wherein the handle portion has a body to which one of the tube and the wire is connected, and a slide member to which the other of the tube and the wire is connected so as to be slidable relative to the body, and wherein one of the body and the slide member includes recess portions disposed linearly at regular intervals while the other of the body and the slide member includes elastic claws fitted into the recess portions.

According to the invention, the elastic claws are intermittently fitted into the recess portions by sliding the slide member relative to the body. Accordingly, the amount of protrusion of the wire from the tip end of the tube can be adjusted suitably. As a result, the basket can be controlled to have a desired size.

### EFFECT OF THE INVENTION

According to the medical treatment tool for tubular organ of the invention, the diameter of the basket can be widened or reduced by the pushing or pulling operation of the handle portion. Accordingly, even when the lumen of the tubular organ or the like is filled with the foreign substances so that a gap of the lumen is small, the basket can be passed through the lumen easily while the diameter of the basket is reduced suitably and then the operation of discharging foreign substances can be performed surely.

Moreover, the basket is formed into a cylindrical shape by knitting and/or braiding plural metal wire rods so that the basket has flexibility compared with a balloon. Accordingly, even if the basket passes through the narrow portion when the medical treatment tool is pulled back after the foreign substances are captured by the basket, diameter-widening force of the basket can be kept while the diameter of the basket is reduced suitably. Accordingly, resistance for pulling out the basket can be reduced, and the foreign substances can be discharged surely and smoothly.

### BRIEF DESCRIPTION OF THE DRAWINGS

FIG. 1 perspectively illustrates an embodiment of a medical treatment tool for tubular organ according to the invention.

FIG. 2A is a sectional view taken in the arrow line X-X in FIG. 1, and FIG. 2B is a sectional view taken in the arrow line Y-Y in FIG. 1.

FIG. 3 is an exploded perspective view of a handle portion of the medical treatment tool.

FIG. 4A illustrates a state where the diameter of a basket is reduced to the minimum, FIG. 4B illustrates a state where the diameter of the basket is widened, and FIG. 4C illustrates a state where the diameter of the basket is widened to the maximum, as an operation of the medical treatment tool.

FIG. 5 is an enlarged view of important part of FIG. 3B.

FIG. 6 illustrates a state where the basket of the medical treatment tool is housed in a catheter.

FIG. 7 illustrates a first use state of the medical treatment tool in which a guide wire is passes through a place where foreign substances are generated.

FIG. 8 illustrates a second use state of the medical treatment tool in which the basket is passes through the place where foreign substances are generated.

FIG. 9 illustrates a third use state of the medical treatment tool in which the diameter of the guide wire is widened so that the foreign substances are captured.

FIG. 10 illustrates a fourth use state of the medical treatment tool in which the foreign substances are discharged to a tubular organ with large diameter.

FIG. 11 is an enlarged view of important part showing another embodiment of the medical treatment tool for tubular organ according to the invention.

FIG. 12 is an enlarged view of important part showing a further embodiment of the medical treatment tool for tubular organ according to the invention.

### DESCRIPTION OF REFERENCE NUMERALS AND SIGNS

10, 10a, 10b medical treatment tool for tubular organ (medical treatment tool)
15 tube
25 wire
30, 30a, 30b basket
31 metal wire rod
33 base end bundle portion
36 tip end bundle portion
35 opening portion
40 handle portion
45 body
50 slide member

### MODE FOR CARRYING OUT THE INVENTION

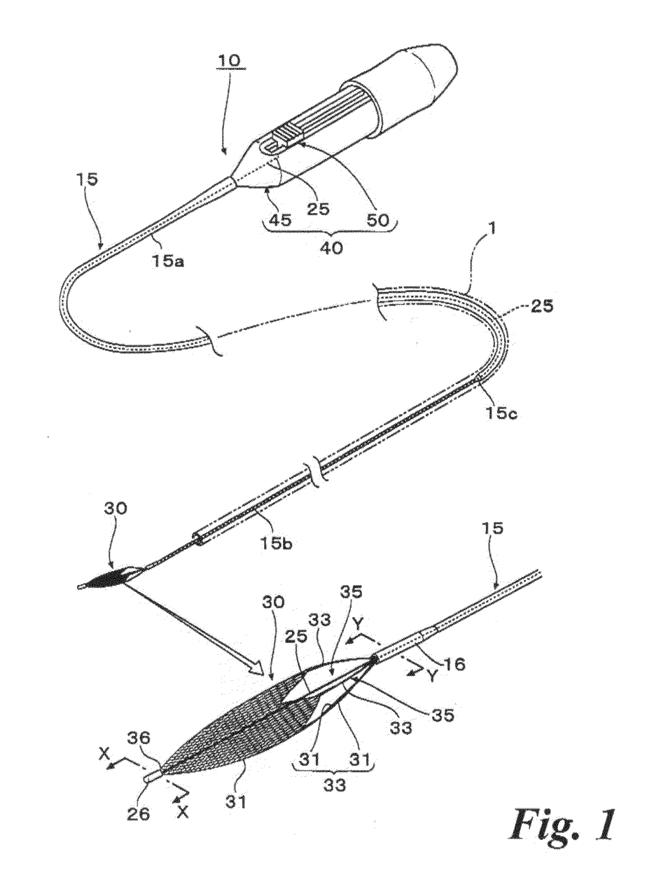

An embodiment of a medical treatment tool for tubular organ according to the invention will be described below with reference to FIGS. 1 to 10.

As shown in FIGS. 1 and 7, the medical treatment tool 10 for tubular organ (hereinafter referred to as “medical treatment tool 10” simply) is inserted into a tubular organ V2, V3, etc. such as a bile duct, a pancreatic duct, etc. for discharging foreign substances G such as gallstones or pancreatic stones generated in the tubular organ, or inserted into a body cavity of a human body for picking and collecting living tissues or the like. As shown in FIG. 1, the medical treatment tool 10 includes: a tube 15; a wire 25 inserted in the tube 15; a basket 30 formed into a cylindrical shape, and having a base end portion connected to a tip end of the tube 15 and a tip end portion connected to a tip end of the wire 25; and a handle portion 40 to which a base end of the tube 15 is connected and which holds a base end portion of the wire 25 to allow a relative movement to the tube 15.

The tube 15 will be described first. The tube 15 is shaped like a hollow cylinder and has a tube base portion 15a which extends by a predetermined length with a fixed outer diameter, and a tube tip portion 15b which is connected to the tube base portion 15a through a taper portion 15c tapering and which extends by a predetermined length with a fixed outer diameter. According to this configuration, the base end side of the tube 15 becomes highly rigid and the tip end side of the tube 15 becomes flexible. Accordingly, pushability of the tube 15 can be kept, the tube 15 can be easily inserted into a tubular organ or the like due to flexibility of the tip end side, and the tube 15 can be further prevented from kinking. Stainless steel is used as the material of the tube 15 in this embodiment. In addition thereto, a shape memory alloy such as a Ni—Ti-based shape memory alloy, a Cu—Al—Ni-based shape memory alloy, a Cu—Zn—Al-based shape memory alloy etc., another flexible metal or resin tube, or the like may be used as the material.

The wire 25 inserted in the tube 15 is made of a Ni—Ti-based shape memory alloy, a Cu—Al—Ni-based shape memory alloy, a Cu—Zn—Al-based shape memory alloy, etc. The wire 25 extends longer than the tube 15, so that when the wire 25 is inserted in the tube 15, the wire 25 protrudes by a predetermined length from the tip end of the tube tip portion 15b of the tube 15 and protrudes by a predetermined length from the base end of the tube base portion 15a of the tube 15.

As shown in FIGS. 1, 4A to 4C and 5, the basket 30 for capturing foreign substances, living tissues, etc. in a tubular organ or a body cavity is disposed on the outer circumference of the tip end portion of the aforementioned tube 15 and wire 25.

The basket 30 is formed into a cylindrical shape by knitting and/or braiding one metal wire rod 31 or plural metal wire rods 31. The basket 30 has a base end portion bundled and connected to the tip end portion of the wire 25, and a tip end portion bundled and connected to the tip end portion of the wire 25, so that opposite ends of the cylinder of the basket 30 are tapered to thereby substantially form a spindle shape. In this embodiment, seventy-two metal wire rods 31 are used for forming the basket 30. Incidentally, the number of metal wire rods 31 for forming the basket 30 is preferably in a range of 24 to 144, more preferably in a range of 36 to 72.

Each metal wire rod 31 in this embodiment is made of a Ni—Ti-based shape memory alloy. In addition thereto, each metal wire rod 31 made of another shape memory alloy such as a Cu—Al—Ni-based shape memory alloy, a Cu—Zn—Al-based shape memory alloy, etc., stainless steel, or the like may be used. The wire 25 may be made of a composite wire rod which includes a radiopaque core made of Pt, Ti, Pd, Rh, Au, W or an alloy thereof, and a coating substance put on an outer circumference of the core and made of the aforementioned shape memory alloy, the aforementioned stainless steel, or the like. A metal wire rod having a diameter of 0.04 mm is used as each metal wire rod 31 in this embodiment. Incidentally, a metal wire rod in a diameter range of 0.02 mm to 0.2 mm can be preferably used as each metal wire rod 31.

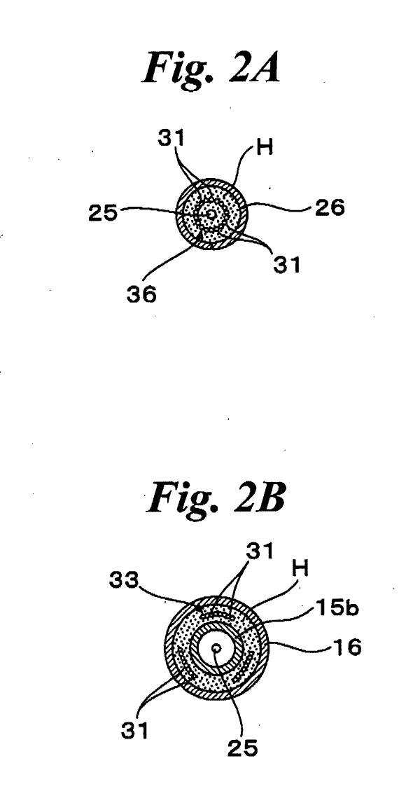

The metal wire rods 31 on the base end portion side of the basket 30 are bundled so that plural (three in this embodiment) base end bundle portions 33 each including plural metal wire rods 31 are formed. The base end bundle portions 33 are disposed at equal intervals on an outer circumference of the tip end portion of the tube tip portion 15b of the tube 15 and joined to the tip end portion of the tube 15 through a base end joint portion 16. That is, as shown in FIG. 2B, the base end joint portion 16 is shaped like a pipe which is put on an outer circumference of the base end bundle portions 33 disposed on the outer circumference of the tube tip portion 15b. A gap between the base end joint portion 16 and the tube tip portion 15b is filled with a connection member H such as wax, solder, an adhesive agent, etc., so that the base end bundle portions 33 of the basket 30 are joined to the tip end portion of the tube 15. Because the base end bundle portions 33 are joined to the tip end portion of the tube 15, opening portions 35 are formed between the base end bundle portions 33.

On the other hand, the metal wire rods 31 on the tip end portion side of the basket 30 are bundled so annularly as to enclose the outer circumference of the tip end portion of the wire 25, so that a tip end bundle portion 36 is formed. The tip end bundle portion 36 is disposed on the outer circumference of the tip end portion of the wire 25 and joined to the tip end portion of the wire 25 through a tip end joint portion 26. That is, as shown in FIG. 2A, the tip end joint portion 26 is shaped like a pipe which is put on the outer circumference of the tip end bundle portion 36. The inner circumference of the tip end joint portion 26 is filled with the connection member H such as wax, solder, an adhesive agent, etc., so that the tip end bundle portion 36 of the basket 30 is joined to the tip end portion of the wire 25.

As described above, the basket 30 has a base end portion connected to the tip end portion of the tube 15 by wax, solder or the like, and a tip end portion connected to the tip end portion of the wire 25 by wax, solder or the like. Alternatively, the pipe-like base end joint portion 16 or tip end joint portion 26 may be pressure-bonded from the outer circumference so as to be joined.

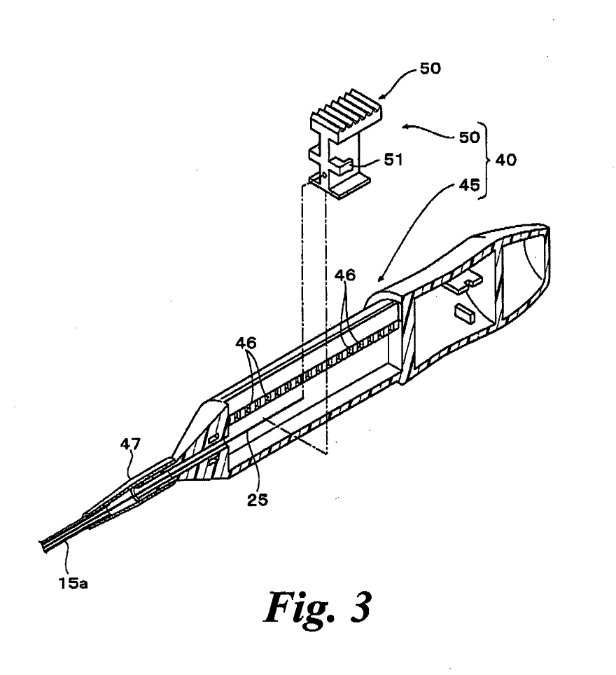

The medical treatment tool 10 is further includes the handle portion 40 which holds the base end portion of the tube 15 and which holds the base end portion of the wire 25 to allow a relative movement to the tube. As shown in FIG. 1, the handle portion 40 has a body 45, and a slide member 50 which is attached to the body 45 so that the slide member 50 can slide. Referring also to FIG. 3, recess portions 46 are provided in inner circumferences of opposite side portions of the body 45 so as to be disposed linearly at regular intervals along an axial direction. Flexible elastic claws 51 are provided from opposite side portions of the slide member 50 so that the elastic claws 51 are fitted into the recess portions 46 in the inner circumferences of the opposite side portions of the body 45. The tube base portion 15a of the tube 15 is connected to the tip end portion of the body 45 through a pipe member 47 while the base end portion of the wire 25 is connected to the slide member 50 so as to be movable relative to the tube 15. By sliding the slide member 50 along the axial direction relative to the body 45, the elastic claws 51 are fitted into the recess portions 46 successively with feeling of clicking so that the amount of protrusion of the wire 25 from the tip end of the tube 15 can be adjusted stepwise.

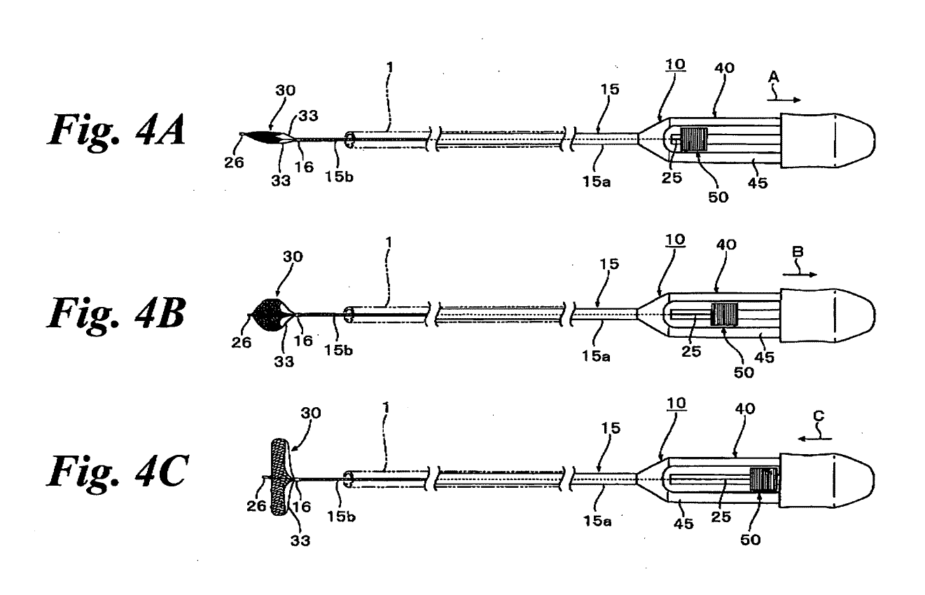

The operation of the aforementioned medical treatment tool 10 will be described below with reference to FIGS. 4A to 4C. By sliding the slide member 50 in the axial direction while holding the body 45 of the handle portion 40, the wire 25 is pushed or pulled so that the diameter of the basket 30 is enlarged or reduced.

FIG. 4A shows a state where the diameter of the basket 30 is reduced to the minimum. When the slide member 50 is slid relative to the body 45 of the handle portion 40 by a predetermined length from this state to the axial-direction base end side (proximal side) as represented by an arrow A, the wire 25 connected to the slide member 50 is pulled back relative to the tube 15 connected to the body 45.

As a result, the portion of the wire 25 protruding from the tip end of the tube 15 is pulled into the tube 15. The base end side of the basket 30 is connected to the tip end portion of the tube 15 while the tip end side of the basket 30 is connected to the tip end portion of the wire 25. Accordingly, the tip end portion of the basket 30 moved in accordance with the pull-in operation of the wire 25 is pulled to the axial-direction base end side so that the basket 30 is pressed and contracted in the axial direction. As shown in FIG. 4B, the diameter of an axial-direction intermediate portion of the cylindrical basket 30 is enlarged in a circumferential direction.

When the slide member 50 is further slid from the state shown in FIG. 4B to the axial-direction base end side (see an arrow B), the portion of the wire 25 protruding from the tip end of the tube 15 is further pulled into the tube 15. Accordingly, as shown in FIG. 4C, the diameter of the axial-direction intermediate portion of the basket 30 is widened largely.

On the other hand, when the slide member 50 is slid relative to the body 45 of the handle portion 40 by a predetermined length from the state shown in FIG. 4C to the axial-direction tip end side as represented by an arrow C, the wire 25 is pushed out by a predetermined length from the tip end of the tube 15. The tip end portion of the basket 30 moved in accordance with the pushing-out operation of the wire 25 is pushed out to the axial-direction tip end side, and the basket 30 is expanded in the axial direction so that the diameter of the basket 30 is reduced again.

By pulling back the slide member 50 relative to the body 45 as described above, the wire 26 is pulled into the tip end of the tube 15, and the basket 30 is pressed and contracted in the axial direction so that the diameter of the basket 30 is widened. On the other hand, by pushing out the slide member 50 relative to the body 45, the wire 25 is pushed out from the tip end of the tube 15, and the basket 30 is expanded in the axial direction so that the diameter of the basket 30 is reduced.

In this embodiment, there is exemplified the case where the slide member 50 is operated to be slid relative to the body 46 of the handle portion 40, the body 45 itself may be slid relative to the slide member 50 as long as the slide member 50 and the body 45 can be moved relative to each other to push or pull the wire 25 from the tip end of the tube 15.

As described above, FIG. 4A shows a state where the diameter of the basket 30 is reduced to the minimum, FIG. 4C shows a state where the diameter of the basket 30 is widened to the maximum, and FIG. 4B shows a state where the diameter of the basket 30 is widened to an intermediate level between the minimum and the maximum. FIG. 5 shows an enlarged view of important part of FIG. 4B. The length L of the basket 30 in a state where the diameter of the basket 30 is widened to an intermediate level is preferably in a range of 5 mm to 30 mm, and the outer diameter D of the basket 30 in this state is preferably in a range of from 3 mm to 30 mm, more preferably in a range of fro 3 mm to 15 mm.

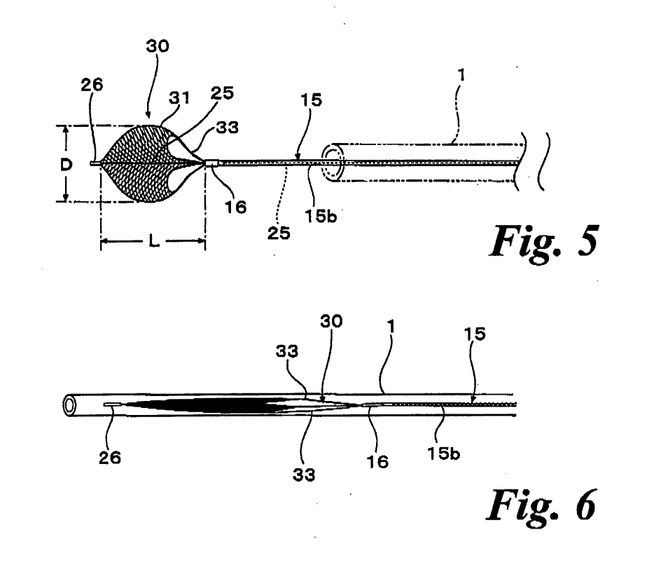

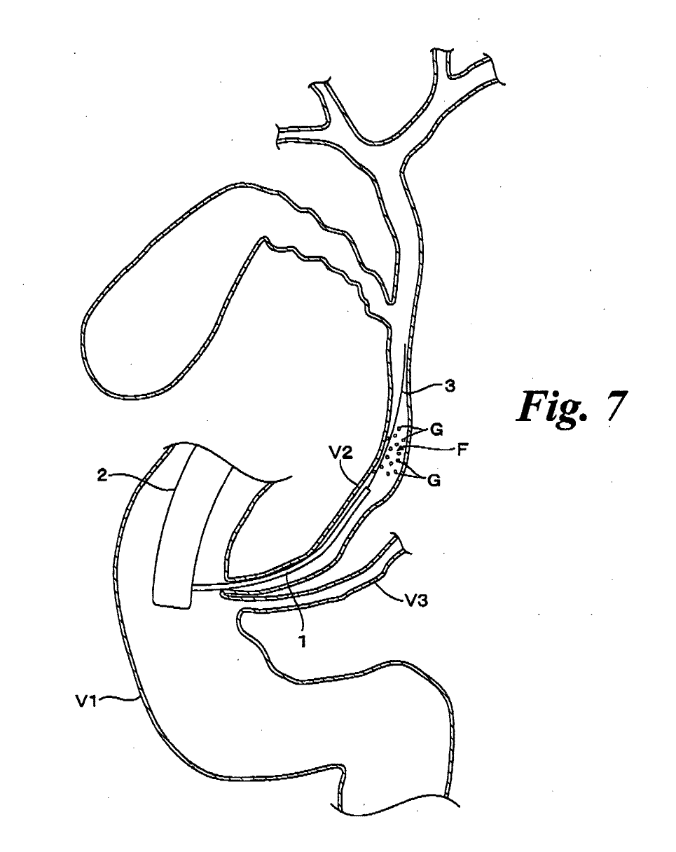

Usage of the aforementioned medical treatment tool 10 of the invention will be described next with reference to FIGS. 6 to 10.

As shown in FIG. 7, for example, the medical treatment tool 10 according to this embodiment can be used for capturing foreign substances G such as gallstones, pancreatic stones, etc. generated in a tubular organ V2, V3, etc. such as a bile duct, a pancreatic duct, etc. and moving the foreign substances G to a tubular organ V1 having a relatively large inner diameter such as the duodenum, etc. to discharge the foreign substances G. Incidentally, the medical treatment tool 10 can be used preferably for capturing foreign substances generated in another tubular organ or living tissues in a body cavity of a human body to discharge or collect the foreign substances or the living tissues.

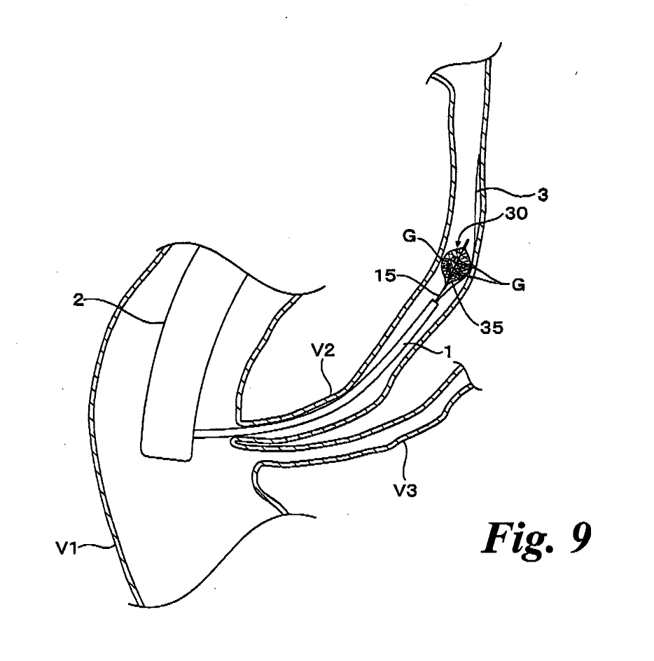

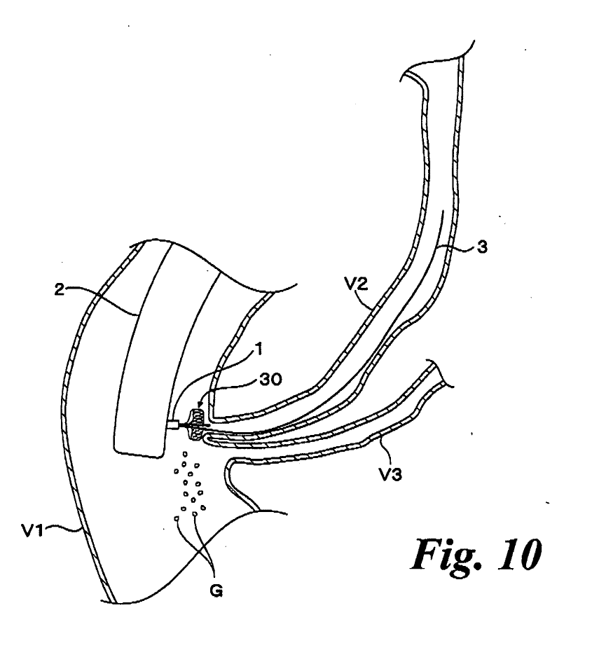

In use, the handle portion 40 is operated as described above to maximum-reduce the diameter of the basket 30 as shown in FIG. 9A, and the medical treatment tool 10 itself is pulled back relative to a catheter 1 (see FIG. 1) externally put on the outer circumference of the tube 15 of the medical treatment tool 10 to house the basket 30 in the inner circumference of a tip end portion of the catheter 1 as shown in FIG. 6.

An example of use of the invention will be described below. First, an endoscope 2 is moved to a large-diameter tubular organ V1 such as the duodenum, etc. through the oral cavity, the stomach, etc. by a well-known method, so that a tip end portion of the endoscope 2 reaches a diverging portion from which tubular organs V2 and V3 smaller in diameter than the tubular organ V1 branch and extend.

Then, a guide wire 3 is introduced through a lumen of the endoscope 2 and passed through a portion F where fragments-like foreign substances G are scattered (hereinafter referred to as “foreign substance scattered portion F”) as shown in FIG. 7 so that a tip end portion of the guide wire 3 reaches a position slightly beyond the foreign substance scattered portion F.

After the guide wire 3 is disposed as described above, a base end portion of the guide wire 3 is inserted into the inner circumference of the tip end portion of the catheter 1 so that the tip end portion of the catheter 1 is externally put on the outer circumference of the guide wire 3. In this state, the catheter 1 is slid along the outer circumference of the guide wire 3 so as to be inserted into the lumen of the endoscope 2.

Then, the catheter 1 is protruded from a tip end opening portion of the lumen of the endoscope 2 and pushed in while visually recognized by the endoscope 2, so that the tip end portion of the catheter 1 reaches a position just before the foreign substance scattered portion F as shown in FIG. 7.

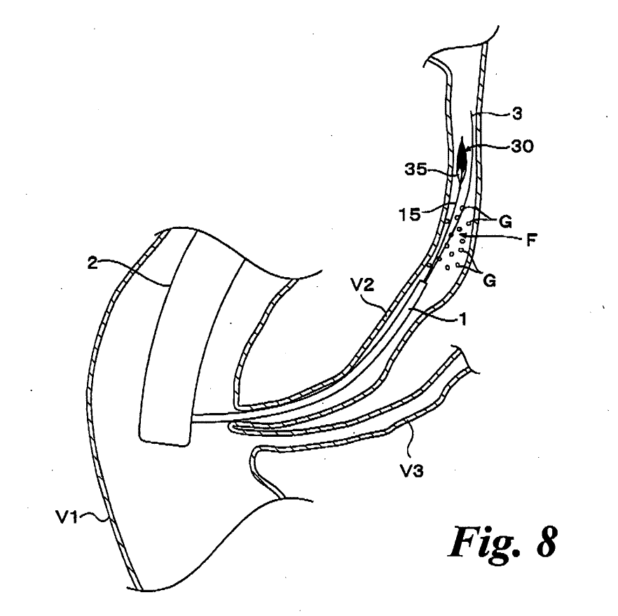

In this state, the medical treatment tool 10 is pushed out as a whole relative to the catheter 1 so that the basket 30 in a reduced diameter state is protruded from the tip end of the catheter 1. By further pushing in the medical treatment tool 10, the basket 30 is passed through the foreign substance scattered portion F and moved to a position slightly beyond the foreign substance scattered portion F as shown in FIG. 8.

The medical treatment tool 10 is configured so that the diameter of the basket 30 can be widened and reduced freely by the pushing and pulling operation of the wire 25 based on the operation of the handle portion 40. Accordingly, the catheter does not have to pass through the foreign substance scattered portion F like the balloon catheter, but only the basket 30 is required to pass through the foreign substance scattered portion F.

When the basket 30 is moved to a predetermined position, the diameter of the basket 30 is widened at that position by the aforementioned operation of the handle portion 40, that is, in such a manner that the slide member 50 is slid to the proximal side relative to the body 45 to pull the wire 25 into the tip end of the tube 15.

The basket 30 in this embodiment is configured so that the opening portions 35 are formed due to the base end bundle portions 33. Accordingly, when the diameter of the basket 30 is widened as described above, the opening portions 35 relatively large are formed on the base end side of the basket 30.

As a result, when the catheter 1 and the medical treatment tool 10 are moved so as to be pulled back to the proximal side in the state where the diameter of the basket 30 is widened, the fragment-like foreign substances G enter into the basket 30 through the opening portions 35 on the base end side of the diameter-widened basket 30 as shown in FIG. 9 so that the foreign substances G can be captured by the basket 30 surely.

In this embodiment, the elastic claws 51 are intermittently fitted into the recess portions 46 with the feeling of clicking when the slide member 50 is slid relative to the body 45 of the handle portion 40. Accordingly, the amount of protrusion of the wire 25 from the tip end of the tube 15 can be adjusted suitably. As a result, the basket 30 can be controlled to have a desired size. Moreover, because the size of the basket 30 can be kept by not sliding the slide member 50, the diameter of the basket 30 can be restrained from being reduced or widened unexpectedly during the push-in or pull-back operation of the medical treatment tool 10.

Moreover, when the catheter 1 and the medical treatment tool 10 are pulled back, the basket 30 is moved to the tubular organ V1 larger in diameter than the tubular organ V2, and the handle portion 40 is operated in this position to repeat the diameter widening/reducing operation of the basket 30 suitably, the foreign substances G caught into the basket 30 can be removed from the basket 30 and discharged into the tubular organ V1 as shown in FIG. 10.

Incidentally, because the basket 30 in the medical treatment tool 10 is formed into a cylindrical shape by knitting and/or braiding plural metal wire rods, the basket 30 has flexibility compared with the balloon inflated with water, air, etc.

Therefore, the basket 30 is fitted to the inner circumferential shape of the tubular organ V1 when the medical treatment tool 10 is pulled back after the foreign substances G are captured by the basket 30. Thus, a gap can be prevented from being generated so that the foreign substances G scattered into the tubular organ V1 can be captured surely. In this embodiment, the basket 30 is made of a Ni—Ti-based shape memory alloy. Accordingly, flexibility of the basket 30 is particularly high so that the basket 30 can be brought into closer contact with the inner circumferential shape of the tubular organ V1 with a less gap as described above.

The root portion of the tubular organ V2 connected to the tubular organ V1 has a relatively small inner diameter. Even when the basket 30 passes through such a narrow portion, diameter widening force of the basket 30 can be kept while the diameter of the basket 30 is reduced suitably in accordance with the inner diameter of the narrow portion. Accordingly, resistance for pulling the basket 30 out can be reduced, and the foreign substances G can be discharged surely and smoothly.

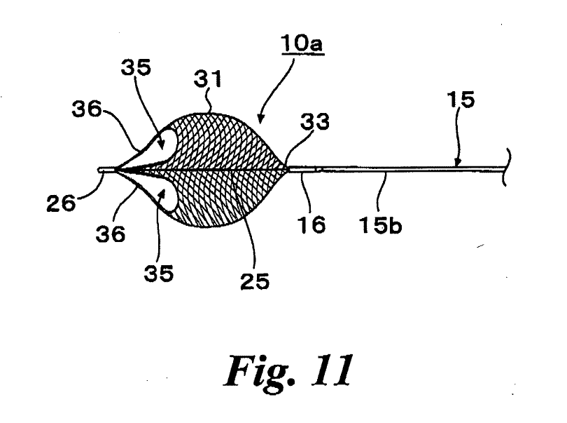

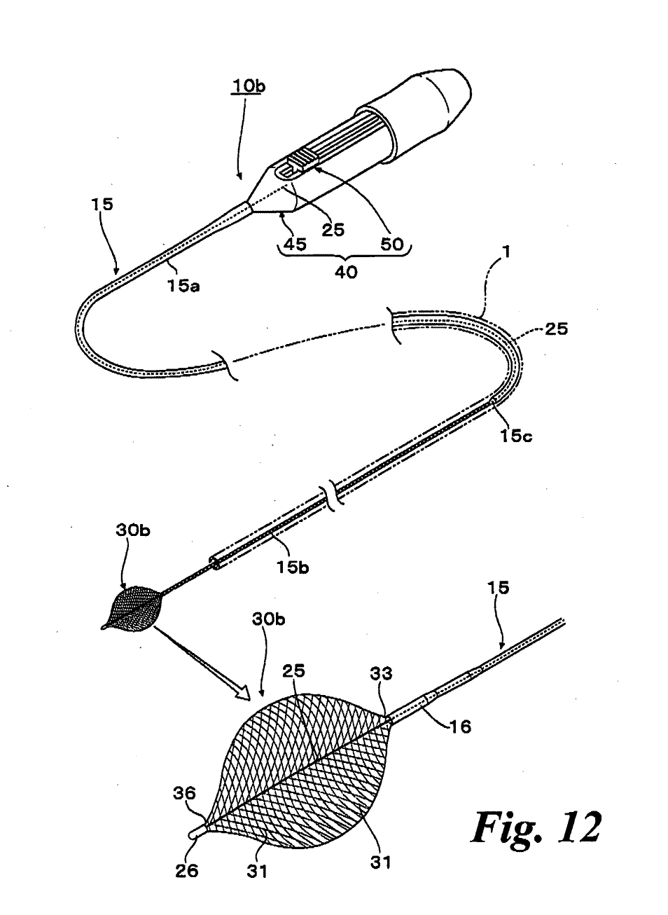

FIG. 11 shows another embodiment of the medical treatment tool for tubular organ according to the invention. Parts substantially the same as those in the aforementioned embodiment are referred to by the same numerals and description thereof will be omitted.

As shown in FIG. 11, the medical treatment tool 10a for tubular organ (hereinafter referred to as “medical treatment tool 10a” simply) is different from the aforementioned embodiment in the positions of opening portions 35 provided in the basket.

That is, metal wire rods 31 on the base end portion side of the basket 30a are bundled to form a base end bundle portion 33 which is jointed to a tip end portion of the tube 15 through a base end joint portion 16. On the other hand, the metal wire rods 31 of the basked 30a at a side of the tip end portion are formed into plural tip end bundle portions 36 which are each formed by bundling plural metal wire rods 31 and which are joined to a tip end portion of the wire 25 through a tip end joint portion 26 so that opening portions 35 are formed between the tip end bundle portions 36. In this manner, the basket 30a in this embodiment has a structure in which opening portions 35 are provided on the tip end portion side of the basket 30a.

In use, after a tip end of the catheter 1 is located just before the foreign substance scattered portion F, the medical treatment tool 10a is pushed out so that the basket 30a in a diameter-reduced state is moved to a position beyond the foreign substance scattered portion F in the same manner as in the aforementioned embodiment. Then, by widening the diameter of the basket 30a and by pulling back the medical treatment tool 10a to the proximal side, the fragment-like foreign substances G are captured to be scraped by the mesh portion of the diameter-widened basket 30a and dragged as they are, so that the foreign substances G can be moved to the tubular organ V1 and discharged.

For example, the foreign substances G may be excessively captured into the basket 30a so that the foreign substances G excessively captured into the basket 30a can be hardly discharged to the tubular organ V1 large in lumen because the basket 30a hardly passes through the narrowed region of the tubular organ V2 or the basket 30a are hardly opened and closed. In this embodiment, the foreign substances G excessively captured into the basket 30a can be discharged from the opening portions 35 by an operation of moving the medical treatment tool 10 back and forth in the tubular organ V2 because the opening portions 35 are provided on the tip end portion side of the basket 30a. As a result, the diameter of the basket 30a can be reduced to a size allowed to pass through even the narrowed region of the tubular organ V2, so that the basket 30a having the foreign substances G captured therein can be passed through the narrowed region smoothly, and that an operation of discharging the foreign substances from the basket 30a in the tubular organ V1 large in lumen can be performed easily.

FIG. 12 shows a further embodiment of the medical treatment tool for tubular organ according to the invention. Parts substantially the same as those in the aforementioned embodiment are referred to by the same numerals and description thereof will be omitted.

As shown in FIG. 12, the medical treatment tool 10b for tubular organ (hereinafter referred to as “medical treatment tool 10b” simply) has a structure in which both tip end portion side and base end portion side of the basket 30b are bundled annularly and joined to the tip end portion of the wire 25 and the tip end portion of the tube 15 respectively without formation of any opening portion 35.

Also in this embodiment, after the tip end of the catheter 1 is located just before the foreign substance scattered portion F, the medical treatment tool 10 is pushed out so that the basket 30b is moved to a position slightly beyond the foreign substance scattered portion F in the same manner as in the embodiment shown in FIGS. 1 to 10. Then, when the diameter of the basket 30b is widened and the medical treatment tool 10 is pulled back to the proximal side, the fragment-like foreign substances G are captured to be scraped by the mesh portion of the diameter-widened basket 30b and dragged as they are, so that the foreign substances G can be moved to the tubular organ V1 and discharged.

1. A medical treatment tool for tubular organ, comprising:
a tube;
a wire comprising a shape memory alloy and inserted in the tube;
a basket formed into a cylindrical shape having a mesh pattern by knitting and/or braiding a plurality of metal wire rods, a base end portion thereof being bundled and connected to a tip end portion of the tube, a tip end portion thereof being bundled and connected to a tip end portion of the wire; and
a handle portion which holds a base end portion of the tube and which holds a base end portion of the wire to allow a relative movement to the tub,
wherein the metal wire rods of the basket at a side of the base end portion or the tip end portion are formed into a plurality of bundle portions each formed by bundling a plurality of metal wire rods, and
wherein the bundle portions are joined to the tip end portion of the tube or the tip end portion of the wire so that opening portions are formed between the bundle portions.

2. The medical treatment tool of claim 1,
wherein the metal wire rods of the basket at the side of the base end portion are formed into the bundle portions each formed by bundling the metal wire rods, and
wherein the bundle portions are jointed to the tip end portion of the tube so that the opening portions are formed between the bundle portions.

3. The medical treatment tool of claim 1,
wherein the metal wire rods of the basket at the side of the tip end portion are formed into the bundle portions each formed by bundling the metal wire rods, and
wherein the bundle portions are jointed to the tip end portion of the wire so that the opening portions are formed between the bundle portions.

4. The medical treatment tool of claim 1,
wherein the handle portion includes a body to which one of the tube and the wire is connected, and a slide member to which the other of the tube and the wire is connected so as to be slidable relative to the body, and
wherein one of the body and the slide member includes recess portions disposed linearly at regular intervals while the other of the body and the slide member includes elastic claws fitted into the recess portions.

5. The medical treatment tool of claim 2,
wherein the handle portion includes a body to which one of the tube and the wire is connected, and a slide member to which the other of the tube and the wire is connected so as to be slidable relative to the body, and
wherein one of the body and the slide member includes recess portions disposed linearly at regular intervals while the other of the body and the slide member includes elastic claws fitted into the recess portions.

6. The medical treatment tool of claim 3,
wherein the handle portion includes a body to which one of the tube and the wire is connected, and a slide member to which the other of the tube and the wire is connected so as to be slidable relative to the body, and
wherein one of the body and the slide member includes recess portions disposed linearly at regular intervals while the other of the body and the slide member includes elastic claws fitted into the recess portions.
